(19) United States
(12) Patent Application Publication
(10) Pub. No.: US 2008/0189820 A1
(43) Pub. Date: Aug. 14, 2008

(54) Protectors

(75) Inventors: John Duffy, East Sussex (GB); John Medforth, Singapore (SG)

Correspondence Address: The BOC Group, Inc., 575 MOUNTAIN AVENUE, MURRAY HILL, NJ, 07974-2082, US

(73) Assignee: THE BOC GROUP PLC, Windlesham, Surrey (GB)

(21) Appl. No.: 11/665,702

(22) PCT Filed: Oct. 12, 2005
(86) PCT No.: PCT/GB2005/003916

§ 371 Date: Dec. 11, 2007

(30) Foreign Application Priority Data
Oct. 22, 2004 (GB) 0423526.3

(51) Int. Cl. A61F 9/06; A42B 1/00; G06F 15/00

(52) U.S. Cl. 2/8.2; 2/424; 702/188

(57) ABSTRACT

A protector for the head, face and/or eyes, for example a welder's helmet, carries at least one gas sensor in a position where the sensor can detect one or more properties of the atmosphere to be inhaled by a person wearing the protector. The gas sensor is operatively associated with at least one data conversion, data transmission and/or data memory device also carried by the protector.

### CROSS-REFERENCE TO RELATED APPLICATIONS

This application claims priority from International Patent Application No. PCT/GB2005/003916 filed Oct. 12, 2005; which claims priority from British Patent Application No. 0423526.3 filed Oct. 22, 2004.

### BACKGROUND OF THE INVENTION

This invention relates generally to protectors for the head, face and/or eyes, and in particular, but not exclusively, to helmets, masks and goggles and other items of headwear that have a protective purpose, particularly but not exclusively for industrial use.

One example of such a protector is a welder's helmet. A welder's helmet fits over the face and forehead and typically has a headstrap which fits around the head and may be adjusted for comfort. The helmet is open at the back but closed at the front so as to protect the welder from molten metal or other hot matter which may be violently ejected from the welding arc as spatter. The front of the welder's helmet has a visor through which the welder can observe the welding arc. The visor is made of a material that does not transmit ultraviolet radiation and that therefore protects the welder's eyes from such radiation.

Spatter and ultraviolet radiation are not, however, the only hazards that a welder faces. Potentially toxic or injurious gases or vapours may be formed in or ejected from the welding arc. These hazards typically go undetected and unrecorded. Analogously, those people carrying out other industrial operations or even going about their normal daily lives may face similar airborne hazards. For example, cyclists are continually exposed to oxides of nitrogen as they ride along suburban and urban roads.

### BRIEF SUMMARY OF THE INVENTION

In its broadest aspect the present invention provides a protector for the head, face and/or eyes which carries at least one gas sensor in a position where the sensor can detect one or more properties of the atmosphere to be inhaled by a person wearing the protector, the gas sensor being operatively associated with at least one data conversion, data transmission and/or data memory device also carried by the protector.

The protector is preferably a helmet or mask, for example, a welder's helmet.

The gas sensor is preferably adapted to detect the concentration of one or more gaseous species in the atmosphere. The gaseous species may, for example, be ozone, nitrous oxide or nitric oxide.

The protector may, if desired, include other sensors. For example, there may be a sensor of particulate material in the atmosphere. Such a sensor can use any of the following measurement techniques: a laser diode method; an electrical method in which a parameter such as electrical resistivity is measured; and a method analogous to any employed in commercial smoke detectors. In a further example, a sensor of electromagnetic radiation, for example, infrared radiation, ultraviolet radiation or blue visible light is included in the protector. In a yet further example, a sensor of noise is included in the protector. The noise sensor typically takes the form of a microphone. In another example an air flow rate sensor is located either on an external or an internal surface of the protector.

The or each sensor is preferably operatively associated with electrical or electronic means for providing instantaneous (“real time”) measurement of a desired parameter and/or for providing cumulative (integrated) measurement of that parameter.

The or each sensor is preferably removable from the protector, typically being a plug-in component.

The protector preferably has a central electronic data processing unit which includes the said data conversion data transmission, and/or data memory device. The central electronic data processing unit typically contains several devices including an analogue to digital signal converter, a programmable comparator to enable signals from the gas sensor to be compared with reference signals and an electrical power source, preferably an electrical battery.

A protector according to the present invention is preferably adapted to operate in one of three different ways. Firstly, the protector may have a data conversion device which converts an input signal from at least one sensor to an output signal which is displayed on a screen carried by the protector. For example, an electronic screen can be incorporated into the visor of a welder's helmet. Alternatively, instead of being visible, the output signal may be audible, for example, an alarm signal. Secondly, the protector may have a data transmission device which transmits an input signal from at least one sensor (converted, if necessary) to a remote station. The signal may be transmitted by any known wireless means, for example by radio frequency telemetry or by so-called Blue Tooth technology. If desired, the protector as well as incorporating a wireless signal transmitter may also incorporate a wireless signal receiver operatively associated with an alarm so that if a signal transmitted to a remote station is found to be indicative of hazardous conditions in the atmosphere being inhaled by a person wearing the protector a warning message or signal can be transmitted back to that person. Thirdly, the data memory device may be arranged to record at chosen intervals values of a particular parameter being monitored by at least one sensor, the data memory device being removable from the protector and loadable from the protector and loadable into a remote data recovery means such as a personal computer. Such an arrangement is particularly advantageous if the operative wearing the protector is to be potentially subjected to a cumulative gaseous hazard. A history if the operative's exposure to the hazard can thereby be gained, and the operative can be removed from duties which present him or her to the hazard should the cumulative measurements show that levels of exposure are approaching those considered to be unsafe.

### BRIEF DESCRIPTION OF THE DRAWINGS

A protector, in the form of a welder's helmet, will now be described by way of example with reference to the accompanying drawings, in which:

FIG. 1 is a schematic front view of the helmet;

FIG. 2 is a schematic of the helmet shown in FIG. 1; and

FIG. 3 is a block diagram illustrating the functions that are able to be performed by a welder's helmet or other protector according to the invention.

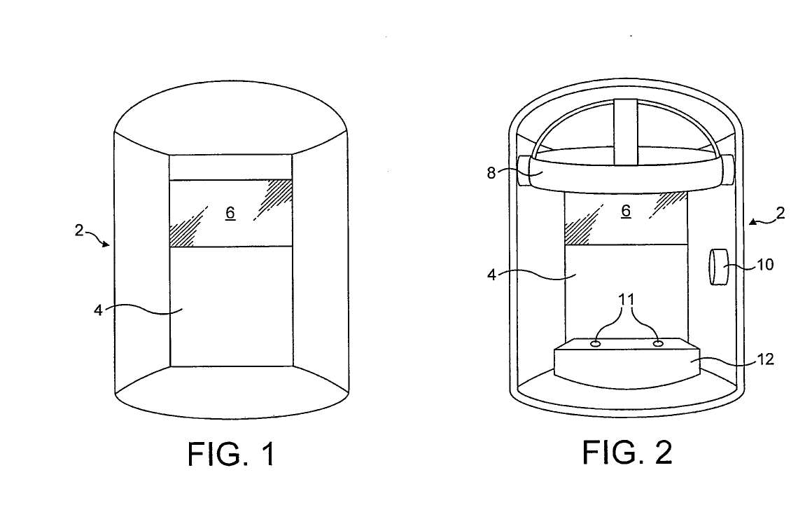

### DETAILED DESCRIPTION OF THE INVENTION

Referring to the drawings, a welder's helmet 2, typically made of a heavy duty plastics material, is closed at the front but open at the rear. The helmet has front face 4 provided with a visor 6 at a height such that it is at eye level when the helmet is worn. The visor 6 is of a material which is transparent to some wavelengths of light in the visible spectrum but opaque to ultra-violet radiation. (Some known visors that are commercially available react to light so as to protect the welder from the intense glare of the welding arc but permit clear visibility otherwise.)

The helmet 2 has an adjustable headstrap 8 to enable a comfortable fit to be made. In accordance with the invention, the helmet 2 carries internally on one of its sides a noise sensor 10. The sensor 10 is in an electrical circuit with a central electronic processing unit which is housed in a box 12 attached to a bottom region of the inner surface of the front face 4 of the helmet 2. The box 12 has further sensors 11 which are adapted to detect the presence of hazardous gases such as ozone and oxides of nitrogen. The sensors 1 are able to generate signals representative of the concentration or partial pressure of one or more hazardous gases in the atmosphere. The hazardous gas may, for example, be ozone, nitrous oxide or nitric oxide. One form of ozone sensor comprises a piezoelectric quartz crystal with a polybutadiene coating. Reaction of the polybutadiene with ozone causes an increase in the mass of the sensor and hence a decrease in oscillation frequency. The rate of change of frequency is proportional to the ozone concentration.

The box 12 contains a data conversion, data transmission and/or data memory device which enable measurements made by the sensor 10 to be transferred either in real time or after the event to a data processing unit which may be included in the box 12 and operatively associated with a display panel or warning device (not shown) carried by the helmet 2 or which may be a at a remote station, signals representative of the measurements being transmitted by a wireless transmitter such as a Blue Tooth device (not shown) or by an emitter (not shown) of radio frequency signals. A further alternative is for the central processing unit to have a removable data memory device which can record the measurements made by the sensor over a period of time and can be subsequently be removed and inserted in a remote processor such as a personal computer.

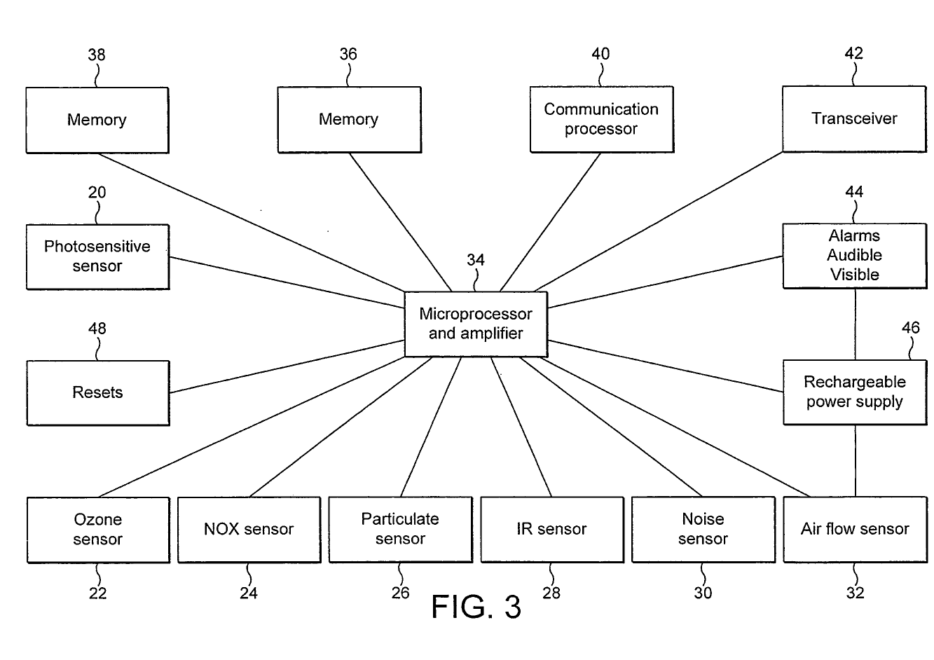

The sensor 10 is preferably reversibly received in a suitable socket (not shown) provided in the helmet 2. The sensors 11 are preferably similarly received in sockets provided in an external surface of the housing of the control box 12. The sockets are connected by appropriate electrical leads (not shown) to the electronic devices in the box 12. If desired, the helmet 2 may comprise an inner shell and an outer shell with the leads to the sensor 10 being located between the two shells. Additional sensors may be provided as will be described below with reference to FIG. 3. Each sensor may be replaceable, for example, by being adapted to be plugged into and removable from a socket.

The signals from the sensors 10 and 11 may be processed in any desired manner either by electronic devices within the box 12 or remotely. Thus, the central data processing unit with the box 12 or at a remote location may include electronic means for differentiating the signals, for integrating the signals, for comparing the signals with programmable reference signals, for generating alarm signals in the event of a hazardous condition being detected, and for displaying instantaneous or cumulative values of the hazardous gas concentration or partial pressure in a graphical form. The central processing unit may employ transducers, analogue-to-digital signal converters, radio frequency wireless transmitted and receivers, random access memory (RAM) devices and the like. The box 12 typically also contains a power source in the form of an electrical battery.

The selection of the components within the box 12 is made so as to avoid adding excessive weight to the helmet 2 that a welder would find uncomfortable. For this reason, embodiments of the invention in which data from the sensor are displayed remotely are often preferred.

The helmet 2 is preferably personal to the welder. A personal record of the welder's exposure to a hazardous gas over a period of time can therefore be built up. Preferably, the helmet carries a plurality of sensors so that the record contains a history of the welder's exposure to a plurality of relevant hazards.

An example of the functions that may be performed by the helmet is now described with reference to FIG. 3. The control box of the helmet has operatively associated therewith an array of sensors comprising a light/ultra violet radiation or photosensitive sensor 20, an ozone sensor 22, a NOx sensor 24, a particulates sensor 26, an infra-red radiation sensor 28, a noise sensor 30 and an internal or external air flow sensor 32. Such sensors are known devices and need not be described herein. They all transmit analogue electrical signals to central microprocessor and amplifier circuits 34. The central microprocessor and amplifier circuits 34 typically include analogue-to-digital signal converters. The central microprocessor and amplifier circuits are also operatively associated with memory devices 36 and 38. The memory devices 36 and 38 may simply be read-write random access memory (RAM) devices. The RAM devices may have associated keep-alive batteries. The memory devices 36 and 38 may be pre-programmed with algorithms relating to each of the parameters being monitored.

The helmet may also be provided with a communication processor 40 to enable information gathered by the sensors to be downloaded to a computer (not shown) and a radio frequency transceiver 42 to enable information to be received by the control box from a remote location or to be transmitted therefrom to a remote location.

The microprocessor circuits 34 are also operatively associated with alarm devices 44 which are adapted to give audible and/or visual alarms in the event of any of the sensors detecting a hazardous condition. The alarm devices 44 are associated with a rechargeable power supply 46, for example, rechargeable batteries. These batteries may be operatively associated with other parts of the system depicted in FIG. 3, for example, the air flow sensor 32. The battery or batteries may be shaped and can be made as part of the helmet casing.

The central microprocessing circuits 34 may also be operatively associated with reset buttons and circuitry 48 that enable cumulative values of selected parameters monitored by the sensors to be reset to zero. The electronic circuits may be incorporated into a single electronic “chip” or a plurality of such “chips”.

1. A protector for the head, face and/or eyes which carries at least one gas sensor in a position where the sensor can detest one or more properties of the atmosphere to be inhaled by a person wearing the protector, the gas sensor being operatively associated with at least one data conversion, data transmission and/or data memory device also carried by the protector.

2. A protector as claimed in claim 2, wherein the helmet is a welder's helmet or mask.

3. A protector as claimed in claim 2, wherein the helmet is a welder's helmet.

4. A protector as claimed in any one of the preceding claims, wherein the gas sensor is adapted to detect the concentration of one or more gaseous species in the atmosphere.

5. A protector as claimed in claim 4, in which the gas sensor is adapted to detect the concentration of ozone, nitrous oxide or nitric oxide in the gaseous atmosphere.

6. A protector as claimed in any one of the preceding claims, wherein the protector additionally carries a sensor of particulate material in the gaseous atmosphere.

7. A protector as claimed in any one of the preceding claims, wherein the protector additionally carries a sensor of electromagnetic radiation.

8. A protector as claimed in any one of the preceding claims, wherein the protector additionally carries a sensor of noise.

9. A protector as claimed in any one of the preceding claims, wherein the protector additionally carries an air flow rate sensor.

10. A protector as claimed in any one of the preceding claims, wherein the data transmission device is a wireless data transmission device.

11. A protector as claimed in claim 10, wherein the wireless data transmission device is a radio frequency data transmission device.

12. A protector as claimed in any one of the preceding claims, wherein the data memory device is removable from the protector and is loadable into a remote data recovery device.

13. A protector as claimed in any one of the preceding claims, wherein the data conversion device includes an analogue to digital signal converter.

14. A protector as claimed in any one of the preceding claims, additionally including a central electronic data processing unit which includes the said data conversion, data transmission or data memory device.

15. A protector as claimed in claim 14, in which the central electronic data processing unit includes a programmable comparator to enable signals from the gas sensor to be compared with reference signals.

16. A protector as claimed in claim 14 or claim 15, in which the electronic data processing unit includes an electrical power source.

17. A protector as claimed in any one of the preceding claims, wherein the gas sensor is removable from the protector.

18. A protector as claimed in claim 17, wherein the gas sensor is a plug-in component.

19. A protector as claimed in any one of the preceding claims, in which the protector has a signal display screen operatively associated with the data conversion device.
